(19) United States
(12) Patent Application Publication
(10) Pub. No.: US 2009/0182330 A1
(43) Pub. Date: Jul. 16, 2009

(54) Bipolar modular foreceps RF voltage conductor assembly

(75) Inventors: Frank D. D'Amelio, Los Olivos, CA (US); Russell J. Redmond, Goleta, CA (US); James W. Cuevas, Santa Barbara, CA (US)

Correspondence Address: Charles W. Hanor, 750 Rittiman Road, San Antonio, TX, 78209, US

(73) Assignee: Live Tissue Connect, Inc., Corpus Christi, TX (US)

(21) Appl. No.: 12/008,435

(22) Filed: Jan. 11, 2008

(51) Int. Cl. A61B 18/14

(52) U.S. Cl. 606/51

(57) ABSTRACT

The bipolar modular forceps assembly is a disposable electrosurgical device utilized with an RF voltage supply to weld soft tissue to prevent bleeding when cut or excised. The device consists of two modularly constructed arms arranged in scissor configuration that clamp when the arms are apart and open when the arms are squeezed together. The wires enter from the rear of the device, behind the digit insertion of the upper arm, and run through the interior of the upper arm before reaching the pivot point. From the pivot joint, one wire continues to the bottom jaw and the other wire wraps around the pin bushing in the pivot joint and proceeds to the top jaw. At all times the wires are fully insulated by the insulative cover with the only exposed wire occurring where they enter the device. The bipolar voltage of each wire is transferred to an electrode on each jaw. The electrodes are insulated from surrounding tissue by the insulative jaw, which contains visible markings showing the beginning, middle and end of the electrode. Both the insulative jaw and the electrode are interchangeable and made from moldable material to allow for different sizes and shapes. When no voltage is applied the tool can be used as a blunt dissector.

### BACKGROUND OF THE INVENTION

1. Field of the Invention

The Bipolar Forceps Modular Assembly is an electrosurgical instrument used which when connected to an RF voltage supplying machine can be used sealing, welding, cutting, coagulation, fusing and transecting tissue. Tissue welding is a smokeless method to prevent bleeding without charring the soft tissue.

RF voltage is utilized to weld soft tissue to prevent bleeding during incisions or excision of soft tissue. The system works through attachment of a bipolar tool to an RF voltage supply. The bipolar tool uses two electrodes of opposite polarity that clamp to each side of soft tissue. When the RF voltage is applied, the soft tissue is heated. Once the tissue has been heated to the appropriate temperature, the result is believed to include the albumin proteins apparently beginning to denature and forming tangled strands, resulting in coagulation. The welded tissue can then be cut without any bleeding occurring due to the coagulation effect of the tangled denatured albumin protein.

2. Description of the Related Art

The prior art includes hand tools that are made of metal and may include some type of insulative or non conductive insulation. Additionally, the prior art includes bipolar hand tools that includes wires that are connected to a power source and to a forceps type tool. The apparatus in US patent publication No. 20070066969A1 utilizes disposable jaw sections with a cable that is connected to opposing electrodes and a separate enclosure assembly attached to the hand piece to insulate the wires.

The apparatus in US patent publication No. 20050101945A1 shows the wires supplying the bipolar RF voltage entering independently through each arm, distinct from the disclosed invention with supplies the wires through one arm.

The apparatus in US patent publication No. 20060064086A1 utilizes a separate jaw assembly that independently attaches to each arm of the forceps. Additionally the apparatus in said invention uses microsealing pads.

### BRIEF SUMMARY OF THE INVENTION

The bipolar modular forceps assembly is a handheld electrosurgical instrument utilized to cut, weld, fuse and coagulate soft tissue, hereinafter referred to as soft tissue welding, and to prevent bleeding when tissue is cut or excised with use of an additional cutting tool. Attached to an RF power supply, the bipolar modular forceps assembly provides for an insulated, effective, safe and inexpensive instrument to facilitate soft tissue welding.

The forceps are in a scissors-like configuration with the ability to be open and closed manually through manipulation of the user's own digits. Each arm is modularly constructed and comprising of opposing structural metal frame members having two pivoted arms and inner and outer cover members for each arm. Each structural metal frame member is partly enclosed by inner and outer cover members, leaving a leading tapered end of the structural frame. An insulative jaw cap attaches to and encloses each leading tapered end, but leaves an exposed portion on the non-clamping side. On the clamping side of each jaw, an electrode is attached and set into a holding cavity in each jaw. Insulated wires carry the bipolar RF voltage to the electrodes through only one arm and are enclosed by one of the sets of inner and outer covers of the forceps and enter from behind the digit insertion ring of the upper arm. The insulated wires run from the rear of the forceps assembly through the upper arm to the pivot joint. One wire passes directly through the arm along the stainless steel structural frame member and is connected to the jaw at the distal end of the arm that initially carried both wires from behind the digit insertion ring. The other wire passes from behind the digit insertion ring, then over or around the pivot joint and pin, through the inner covers and over to the leading edge of the stainless steel structural frame member of the other arm. Once past the pivot joint each wire carries electrical RF voltage to the opposing jaw members and to the electrodes attached to each jaw member.

The electrodes are part of the electrically insulative jaw cap system and when not actuated by a bipolar RF voltage can be used as a blunt-end dissector. When clamped, the two jaws grasp the soft tissue. Once actuated, the voltage runs between the two conductive electrodes on both jaws and into the soft tissue to treat it. The insulative jaw caps contain markings show the operator the beginning, middle, and end of the electrode to ensure the exact location of tissue is being welded. Using a tapered jaw, or other useful shape, the forceps may be used as blunt dissectors by inserting the front end in the closed position into a gap or opening in the tissue and using the non-clamping side of each jaw to separate and dissect the tissue.

The bipolar modular forceps assembly is inexpensive to make and as such can be used as a disposable device. The modular construction of the device allows for interchangeable parts such as increased handle size or different electrode configurations.

### BRIEF DESCRIPTION OF THE SEVERAL VIEWS OF THE DRAWINGS

FIG. 1 is a side profile view of the invention with the jaws in the closed position.

FIG. 2 is a bottom profile view looking up at the lower arm of the invention.

FIG. 3 is a composite drawing of the modular construction of the invention as viewed from the side.

FIG. 4 is a cross sectional view of the invention taken from the perspective of the pivot joint viewing towards the jaws.

FIG. 5 is an angular cross sectional view showing the wire configuration around the pivot joint.

FIG. 6 is a cross sectional view of the invention taken from the perspective of the interchangeable modular jaw members viewing towards the pivot joint.

FIG. 7a is a bottom side perspective view of the electrode in one embodiment.

FIG. 7b is a top side perspective view of the electrode.

FIG. 8 is a top side angular view of the electrode in an alternative embodiment showing a cutting member.

FIG. 9 is a perspective view of the stainless steel structural frame member showing the passage of the RF voltage carrying wires along the elongated portion of the frame.

FIG. 10 is a cross-sectional view of the upper arm showing the connection between the wires and the connector.

FIG. 11 is a composite drawing of the construction of the connector.

FIG. 12a is a top side perspective view of the interchangeable modular jaw member.

FIG. 12b is a bottom side perspective view of the interchangeable modular jaw member.

FIG. 13a is a cross sectional perspective view of the wire side plug

FIG. 13b is a perspective view of the non-wire side plug.

### DETAILED DESCRIPTION OF THE INVENTION

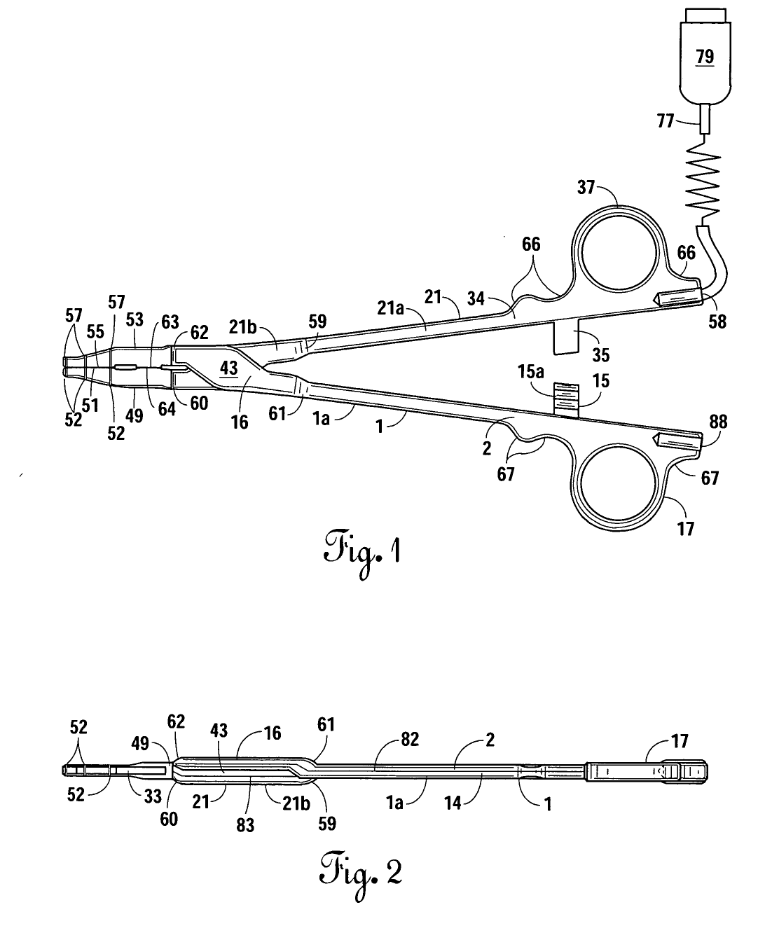

FIG. 1 illustrates the side view structure of the bipolar modular forceps assembly. In an opposing scissors-like configuration, the upper arm member 21 and lower arm member 1 meet at pivot joint 43, located proximal to the front of the forceps. Upper arm member 21 features a digit insertion ring 37, finger grooves 66 and a first opposing jaw member 49 at extreme opposite ends with an elongate shaft 21a connecting the two functional features; the pivot joint 43 is proximally located to the bottom jaw member 49. The first opposing jaw member holds a first opposing electrode in a holding cavity. Also, the upper arm member is comprised of a first and second opposed elongated cover members which partially enclose a first opposed structural frame member scissor arm. Lower arm member 1 features a digit insertion ring 17, finger grooves 67 and a second opposing jaw member 53 at extreme opposite ends with an elongate shaft 1a connecting the two functional features; the pivot joint 43 is proximally located to the top opposing jaw member 53. The second opposing jaw member holds a second opposing electrode in a holding cavity. Also, the upper arm member is comprised of a third and fourth opposed elongated cover members which partially enclose a second opposed structural frame member scissor arm. The first, second, third and fourth cover members are made of moldable material. The cover members, however, can but need not be made of insulative material. The pivot joint 43 connects the upper arm and lower arm together. Connected to the upper arm is a cable 77 and connector 79 which can be attached to an RF voltage source to power the electrodes.

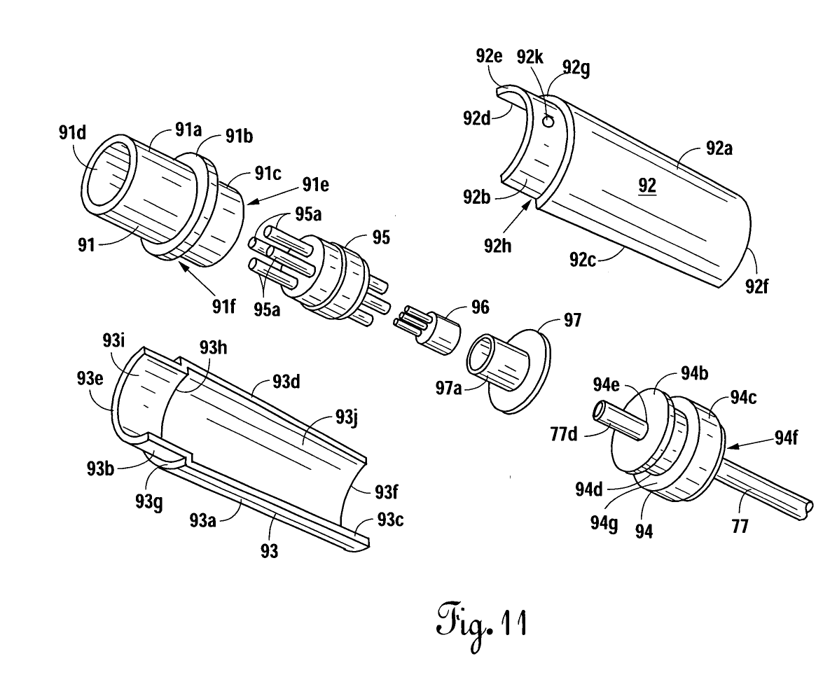

In the clamped but unlocked configuration as shown in FIG. 1, the bottom opposing jaw member 49 and top jaw member 53 clamp at two places; the first clamping point is between the clamp stops 64 and 63 and the second is between the electrodes, 51 and 55. Clamp stops 64 and 63 on the bottom opposing jaw member 49 and top opposing jaw member 53, respectively, provide a dead stop located at one end of each jaw, proximal to pivot joint 43. Textures, 64d and 63d respectively, of clamp stops 64 and 63 are roughened for added friction (see FIGS. 12 and 12b). The second clamping point occurs at the opposed end of the jaws, distal to pivot joint 43, at the two electrodes 51 and 55 located on top opposing jaw member 53 and bottom opposing jaw member 49, respectively. In the clamped configuration, with the two clamp stops 64 and 63 touching, the digit insertion ring 37 on the upper arm 21 and digit insertion ring 17 on the lower arm 1 are at the nearest point that these stops allow. Regulated by the height of the clamp stops, a gap of about 0.0025 to 0.006 inches is maintained between the two electrodes 51 and 55 so that they do not touch each other when the tool is closed. The clamp stops can be molded to increase or decrease the gap between electrodes by raising the height of the stop height 63b (shown in FIG. 12b) and 64b. It is a preferred practice, however, that the electrodes should not directly touch each other. To open the jaws of the bipolar modular forceps assembly, the operator's digits, inserted in the upper arm 21 at digit insertion ring 37 and lower arm 1 at digit insertion ring 17, are spread apart to pivot both arms further apart and open the bottom jaw 49 and top jaw 53. Arm stops, comprised of members 68, 68a, 69, and 69a, are built into the inner portions of the upper and lower arm to prevent over separation of the two arms and the bottom and top jaws. In the open configuration, tissue can then be placed in between the jaws and subsequently clamped by the operator squeezing the rings together, in the opposite manner of opening the bipolar modular forceps assembly. The ratchet teeth 35a on the locking member 35 on the upper arm 21 mate with ratchet teeth 15a on the locking member 15 on the lower arm 1 to form a releasable locking mechanism that allows the jaws to be locked together by closing the forceps and without having to maintain force on the forceps. The locking mechanism is overcome through the operator exerting downward and side-way pressure pulling the two locking members 15 and 35 laterally apart so that the ratchet teeth 35a disengage from the corresponding ratchet locking teeth 15a. This locking mechanism is standard on many forceps.

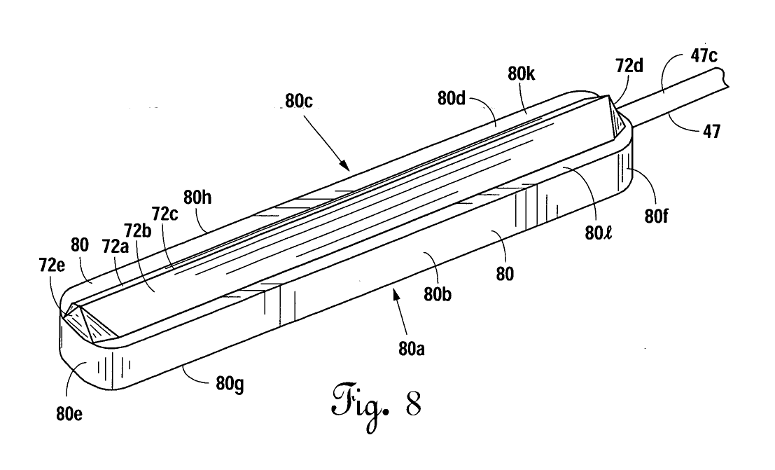

The use of opposing interchangeable modular jaw members and opposing interchangeable electrodes allows for the attachments of various shaped jaws and electrodes on the tool. For instance, the electrodes on the jaws could be sized and shaped for various forms of tissue treatment (see FIGS. 6 and 8). Various sizes and shapes of jaws and electrodes could be used. Because the jaws may be moldable plastic or other insulative material the desired shape for a particular type of surgery can be used in combination with the other common components of the tool. The jaws could be various shapes such as a platypus, square, rectangular, circular, and oval depending on the type of surgery.

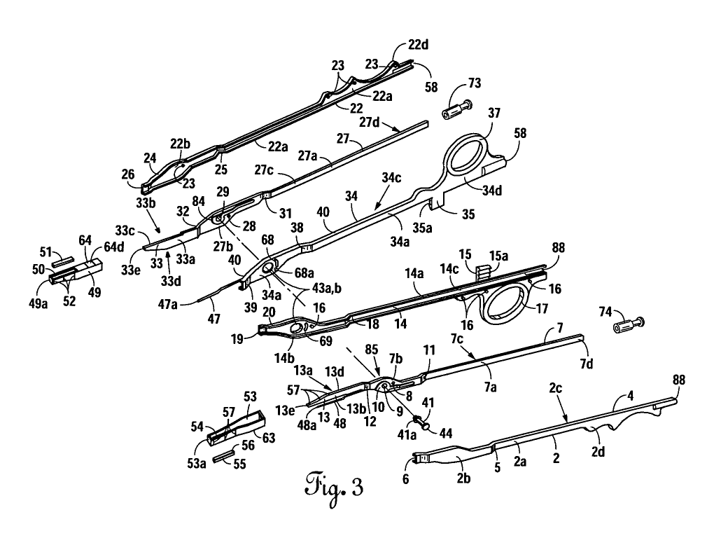

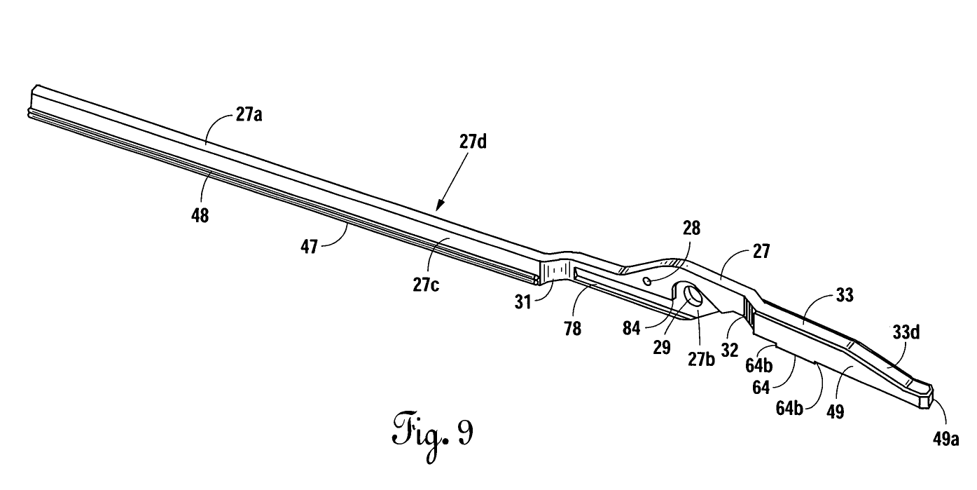

When not used as a tissue treatment device, the apparatus can be used as a tissue dissector. The embodiment shown in FIG. 1 shows the jaw members 53 and 49 having a tapered end. Also, FIGS. 2 and 9 show that the non-clamping side of each jaw member has an exposed leading edge 33d and 13d (shown in FIG. 3) of the stainless steel structural frame members, 27 and 7, respectively. These exposed leading edges provide a dissection surface. Utilizing the pointed ends 52a and 49a, the apparatus can be leveraged into an opening in tissue and dissected by opening the scissor arms. This widens the tissue opening and facilitates access to underlying tissue.

FIGS. 12a and 12b illustrate the opposing interchangeable modular jaw member construction. The modular jaw members are constructed from insulative material to insulate and shield each exposed wire from the stainless steel structural member and the user. This construction electrically isolates the electrode from the frame and the user without the necessity of using covers made from insulative material. The opposing interchangeable modular jaw member is comprised of a front face 53a, side external walls 53b and 53c which continue from the front face to the arm connecting end 53g of the jaw, an electrode holding cavity 50, bottom surfaces 53j and 63c, a clamp stop 63, and the clamp stop bulge 63a. The internal portion of the modular jaw is comprised of a channel member 53k with channel walls 53e and 53d and a channel bottom surface 53h. The clamping side of the jaw member is the side comprising the electrode holding cavity 50, the bottom surfaces 53i and 53j, and the clamp stop 63. The non-clamping side is the side comprising frame channel 53k where the stainless steel structural frame member slides in and forms top exposed leading edge 13d (not shown). This opposing modular jaw member accepts the structural frame member into the channel member, accepts the electrode into the electrode holding cavity, and accepts one RF voltage wire into the wire entrance.

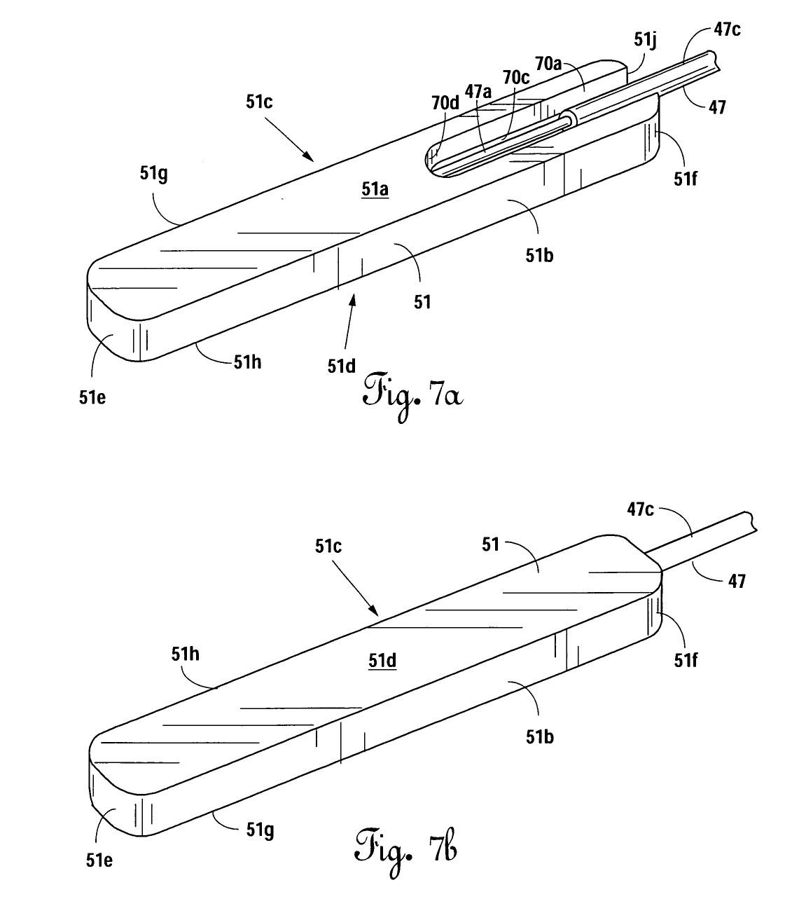

The desired shape of the electrode and the shape of the structural frame member leading edge factor in to shape the modular jaw member. The electrode holding cavity is shaped to accept the electrode and one wire that runs from the stainless steel structural frame member. The electrode holding cavity shown in FIG. 7a is comprised of a front wall 50d, side walls 50b and 50c, a bottom surface 50a, and a wire opening 50e. The front wall and side walls are set into the jaw at edge line 53n which has a shaped to allow the electrode to snugly rest in the jaw to a depth corresponding to the height of front and side walls. This height is set to allow the electrode to protrude a desired amount and also to provide enough surface area to bond the electrode to the walls of the cavity. The intersection of the side and front cavity wall and the bottom surface form bottom edge line 50f. At the wire entrance 50e, located at the distal end from the front wall, the cavity is open to the interior of the modular jaw. This entrance allows the wire carrying RF voltage to enter the cavity and connect to the electrode. The edge line 53n and shape of the electrode determine the shape of jaw bottom edge line 53o. This edge line extends from the arm-connecting portion 53g along the bulging portion 63a, up towards the electrode holding cavity, along the contour of the edge line 53n, and then back to 53g. The purpose of the bulging portion 63a is to allow space within the jaw member to receive the conductor wire as it passes from the stainless steel member to the wire entrance. In turn, the modular jaw front face and side external walls extend vertically from the edge line 53o. The height of these walls is variable to allow for different shapes to the opposing interchangeable modular jaw member.

In its preferred embodiment, the electrode is molded from a conductive material. FIGS. 7a and 7b illustrate the construction of electrode 51. The electrode's shape can be variable to allow for different bonding and cutting scenarios. The electrode 51 is comprised of a top face 51d, a top face edge line 51h, side walls 51b and 51c, a front face 51e, a back face 51f, a bottom face 51a, and a wire inlay 70. The shape of the bonding surface can be varied by reshaping the bonding surface edge 51h. The side, front and back walls extend perpendicularly from this at a desired height to allow the electrode to attach in the electrode holding cavity. Except at the wire inlay portion of the electrode, the bottom edge line 51g follows the shape of the top edge line and forms the bottom face. The bottom edge line 51g is broken by the top cavity edge line 51i which forms the top of an inlay 70 into the solid electrode. The inlay is comprised of side walls 70a and 70b, a front wall 70d and a bottom surface 70c. These elements form the inlay opening 51j which is wide enough to house wire 47 and attach the exposed portion 47a so that it touches the electrode and conducts electrical current.

FIG. 8 shows an alternative embodiment of the interchangeable electrode. The electrodes are interchangeable to allow for different surgical and medical techniques. In this embodiment the topography of the electrode 80 been varied by adding a cutting member 72 on its top face 80d with top face ledges 80l and 80k. The cutting member is comprised of side cutting faces 72a and 72b, a front face 72e, a back face 72d, and cutting edge 72c. The cutting faces 72a and 72b may be angled from the horizontal top face 80d and the outer ledges 80l and 80k may be reduced in width to increase the cutting angle and cutting surface of the member.

FIG. 6 illustrates the assembly of the opposing modular jaw member 53 with electrode 81. The opposing modular jaw member 53 fits into the leading tapered edge 13 of the opposing structural frame member scissor arm 7 such that the leading edge fits fully into the channel member 53k and the front face of the leading edge 13e meets inner front face 53p (not shown). The channel walls 53e and 53d are bonded to the outer walls 13a and 13b of the stainless steel leading edge 13, forming boundary 53l. The bottom surface of the channel 53h is bonded to the bottom surface of the stainless steel leading edge 13c. The top face of the leading edge 13d is left exposed. The wire 48 at its exposed end 48a enters the wire entrance 50e of the electrode cavity 50 such that the wire lies on the bottom surface 50a of the electrode cavity. Before bonding the electrode 81 to the electrode cavity, the exposed wire 48a is attached in the wire inlay 56 of the electrode. Then, the electrode's non-clamping surface 81a is set to face and bonded to cavity bottom surface 50a such that electrode cavity walls 50b, 50c and 50e (front wall) can be bonded to electrode side walls 81b, 81c and 81e (front surface).

The marking lines 52 are shown on the side wall of bottom jaw 49 and indicate the beginning and end of the electrode 51 and a heavier marked line signifying the center of the electrode 51. The marking indicator lines are displayed on both side walls of bottom jaw opposing modular jaw member 49 and on the exposed leading forward edge 33d of the opposing structural frame member scissor arm 27 that displays to the bottom of the invention (shown on FIG. 2). A corresponding set of marking indicator lines 57 are displayed on top opposing modular jaw member 53 and leading forward edge 13 of the opposing structural frame 7 shown in FIG. 3. The marking indicator lines are useful to the operator so they can orient themselves as to the location of soft tissue being welded by the invention.

Each scissor-like arm of the forceps is comprised of two mated cover members partially enclosing a structural metal frame member. In its preferred embodiment, the structural metal is stainless steel. Lower arm 1 comprises a first elongated cover member 2 which is mated and connected to second elongated cover member 14, see FIG. 3, fully enclosing a first stainless steel structural frame member 7, except at the tapered leading edge 13, as shown in FIGS. 1, 2 and 4. The top opposing modular jaw member 53 at 53f is also attached to the tapered leading edge 13, leaving the stainless steel structural frame member 7 exposed at 13d (see FIG. 6). As shown in FIG. 2, the lower arm 1 also includes digit insertion ring 17 and finger grooves 66 located at the end distal from the modular jaw member. The lower arm is also comprised of a cylindrical entrance 88 which is plugged by the non-wire arm plug 74. Upper arm 21 comprises a third opposed elongated cover member 22 which is mated and connected to a fourth opposed elongated cover member 34, fully enclosing a second stainless steel structural frame member 27, except at the tapered leading edge 33. The bottom opposing modular jaw member 49 at 49f (not shown) is also attached to the tapered leading edge 33, leaving the stainless steel structural frame member exposed 33d. The upper arm 21 includes the digit insertion ring 37 and finger grooves 67 located at the rear of the device.

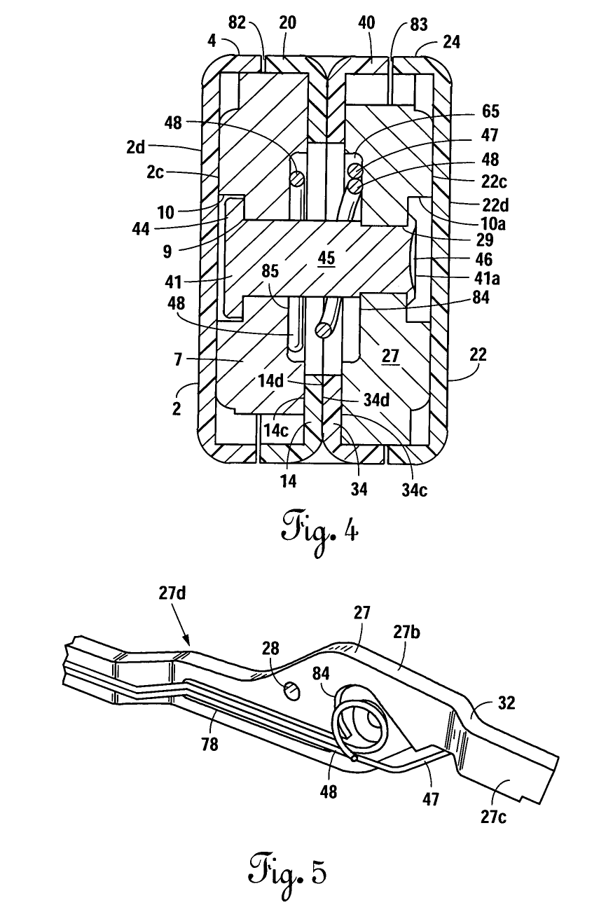

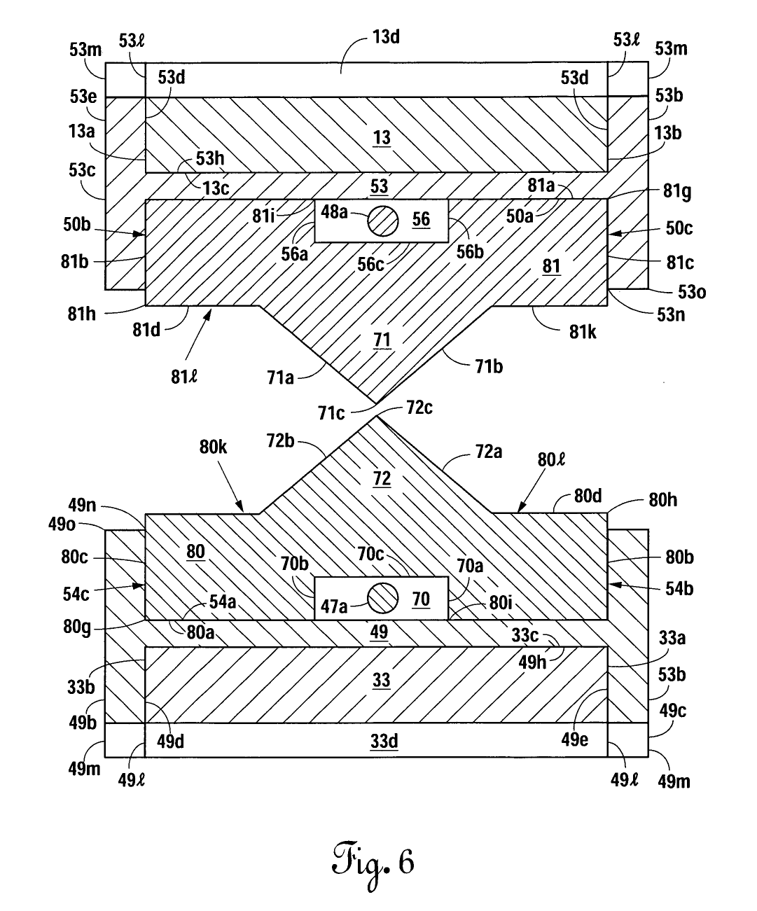

On the lower arm 1, the elongate shaft 1a extends from the digit insertion ring 17 to the bulging offset portion 1b, which contains, in part, pivot joint 43. Middle jutting portion 61 initiates the bulging offset portion distal to top jaw 53 and cap jutting portion 62 ends the bulging offset portion proximal to the top jaw 53 by returning the top jaw 53 to the same plane as the elongate shaft 1a. The bulging offset portion 1b is broader than the elongate shaft 1a in order to encompass the pivot joint 43 and the bipolar wires 47 and 48 (shown on FIGS. 4 and 5). On the upper arm 21, the elongate shaft 21a extends from the digit insertion ring to the bulging offset portion 21b (shown on FIG. 1), which contains pivot joint 43. Middle jutting portion 59 initiates the bulging offset portion 21b distal to bottom modular jaw 49 and cap jutting portion 60 ends the bulging offset portion proximal to the bottom modular jaw 49 by returning the bottom jaw to the same plane as the elongate shaft 21a. The bulging offset portion 21b is broader than the elongate shaft 21a in order to encompass the pivot joint 43 and the bipolar wires (shown on FIGS. 3, 4 and 5). Additionally, the bulging offset portion, created by the middle and cap jutting portions, help establish both the upper arm 21 and lower arm 1 in the same vertical plane at the point of the digit insertion rings 17 and 37 and at the clamping point which assists in user-friendly operator control.

The pivot joint 43 lays in the middle of the offset bulging portion of the upper arm 21 and the lower arm 1. These bulging offset portions 21b and 1b lie parallel to the horizontal plane formed by the elongate shafts 21a and 1a; however these bulging offset portions have been offset from the elongate shaft horizontal plane to accommodate the pivot joint 45. The middle jutting portion 61 of the lower arm 1 extends the elongate shaft 1a 45 degrees away from the horizontal plane (see FIG. 2). The lower arm continues horizontally through the pivot joint 43, as the bulging offset 1b, and then extends 45 degrees through end jutting portion 62 back towards the horizontal plane and to top jaw 53 (shown on FIG. 1), returning the jaw 53 to the same horizontal plane as the elongate shaft and digit insertion ring of the lower arm. The middle jutting portion 59 of the upper arm 21 extends the elongate shaft 21a 45 degrees away from the horizontal plane (see FIG. 2). The upper arm continues horizontally through the pivot joint 43, as the bulging offset 21b, and then extends 45 degrees through end jutting portion 60 back towards the horizontal plane and to bottom jaw 49, returning the jaw 49 to the same horizontal plane as the elongate shaft and digit insertion ring of the upper arm.

Referring to FIG. 3, there is shown an exploded view of the components of the bipolar modular forceps assembly. The lower arm 1 (shown in FIG. 1) comprises an elongated cover member 14, elongated cover member 2. The inner elongated cover member 14 may be a solid piece of molded material that comprises digit insertion ring 17, locking member 15, finger grooves 66, elongated shaft 14a, a circular opening that serves as part of the pivot joint 43, bulging portions 18, 14b, and 19, and lip 20. The lip 20 creates a U-shape with the legs of the lip 20 facing outwards and extending perpendicular from the inner surface 14c. The U-shaped lip 20 extends the length of inner elongated cover member but does not encompass the digit insertion ring 17 but instead continues along the path of the elongated shaft 14a to the cylindrical opening 88 below digit insertion ring 17. Molded cylindrical hollow female receptors 16 are located in the inner surface 14c, with three located near the digit insertion ring and one in the bulging portion 16b. The outer elongated cover member 2 is molded material with elongated shaft 2a that extends to the bulging offset portion 2b. It also features a lip 4 that creates a U-shape with the lip facing inwards towards the lip 20 on inner cover member 14. The outer elongated cover member 2 includes male cylindrical hollow female receptors 3 (not shown) on the inner surface 2c (not shown) that extend in the same manner as the lip 4 and correspond to cylindrical hollow female receptors 16. The singular stainless steel structural frame member 7 of the lower arm 1, fits within the lip 20 and lip 4, and features inlet openings 8 spaced along the frame to correspond with the female joint receptors 16 on the inner surface 14c of inner elongated cover member 14. In the bulge section 7b of the stainless steel structural frame member 7 is the entrance to pivot joint 9 which is surrounded on the exterior face with pin countersink 10. The pin 41 passes through the entrance to pivot joint 9. The outer cover member 2 of the upper arm is fastened to the apparatus by snapping the male connectors of the outer cover member 2 into the cylindrical hollow female receptors 16 on the inner cover member 14 inserting connecting directly to the male cylindrical pin connectors 3 on outer cover member. The lip 20 on the inner cover member 14 touches the lip 4 on inner cover member 2 forming seam 82 with the end result of stainless steel structural frame member 7 being fully enclosed by the inner cover member 14 and outer cover member 2 and having outer surfaces 14d and 2d.

The bulging offset portion 1b of the lower arm 1 comprises the bulging offset portion 14b of the inner cover member 14, 7b of the stainless steel structural frame member 7, and 2b of the outer cover member 2. The middle jutting portion 61 (shown on FIG. 1) comprises middle jutting portion 18 on the inner cover member 14, middle jutting portion 11 on stainless steel structural frame member 7, and middle jutting portion 5 on outer cover member 2 (shown on FIG. 3). The end jutting portion 62 (shown on FIG. 1) comprises end jutting portion 19 on inner cover member 14, end jutting portion 12 on stainless steel structural frame member 7, and end jutting portion 6 on outer cover member 2 (shown on FIG. 3). Inner cover member 14 and outer cover member 2 both terminate at the jaw connection point 62a (not shown) at the end of jutting portion 62. The stainless steel structural frame member 7 continues through the end jutting portion 62, emerging from the enclosed cover member as the leading tapered edge 13.

The leading tapered edge 13 of stainless steel structural frame member 7 features a platypus tapered profile with marking lines 57 displayed at its exposed end 13d and clamp stop 63 that when clamped touches bottom jaw 49. The insulative top jaw 53 slides over the tapered leading edge 13 and abuts the cover (comprised of outer cover 14 and inner cover 2) at end jutting portion 62 (shown on FIG. 1). The top jaw 53 is open at the top, exposing the leading tapered edge 13d with marking indicator lines 57 showing (not shown). On the clamping side of top jaw 53, where it meets bottom jaw 49, is an electrode 55. The electrode 55 inserts into the top jaw 53 in cavity 54 (not shown). The electrode 55 has an inlay 56 (not shown) where wire 48 attaches and touches it, providing to the electrode RF voltage. The marking indicator lines 57 correspond to the beginning, middle, and end of the electrode 55. The marking indicator lines 57 are located on both side walls of the top jaw 53 and on the top of the leading tapered edge 13d of the stainless steel structural frame member 7.

The upper arm 21 as shown in FIG. 3 comprises elongated cover member 34, elongated cover member 22 and enclosed stainless steel structural frame member 27. The inner cover 34 is a solid piece of molded material that comprises a digit insertion ring 37, a locking member 35, finger grooves (figure numbers), elongated shaft 34a, a circular opening that serves as the pivot joint 43, bulging portions 38, 34b, and 39, and lip 40. The U-shaped lip 40 extends the length of the inner elongated cover member but does not encompass the digit insertion ring 37 but instead continues along the path of the elongated shaft 34a to the cylindrical opening 58 below digit insertion ring 37. Molded cylindrical hollow female receptors 36 (not shown) located in the inner surface 34c (not shown) and protrude out in the same direction as the lip 40. The outer elongated cover member 22 is a singular molded piece with elongate shaft 22a that extends to the bulging offset portions 25. It also features a lip 24 that creates a U-shape with the lip facing inwards towards the lip 40 on insulative inner cover 34. The outer elongated cover member 22 includes male cylindrical pin connectors 23 on the inner surface 22c that protrude in the same manner as the lip 24 and correspond to cylindrical hollow female receptors 36. The singular stainless steel structural frame member 27 of the upper arm 21 fits within the lip 40 and lip 24. In the bulge section 27b of the stainless steel structural frame member 27 is the exit 29 to pivot joint. The pin 41 passes from the opening 43a of the inner cover member 34 and then through the exit to the pivot joint 29, passing from frame member side 27c to 27c. The pin's shoulder 41a passes through opening 29 and holds to the inner cover members 14 and 24 together with the stainless steel structural frame members 7 and 27. The outer cover member 22 of the upper arm is fastened to the apparatus by snapping the male connectors 23 of the outer cover member 22 into the female joint connectors 36 on the insulative inner cover 34. The lip 40 on the insulative inner cover 34 touches the lip 24 on insulative inner cover 22 forming seam 83 with the end result of stainless steel structural frame member 27 being fully enclosed by the inner insulative cover 34 and outer insulative cover 22 and having outer surfaces 34d and 22d.

The bulging offset portion 21b of the upper arm 21 comprises the bulging offset portions 34b of the outer cover member 34, 27b of the stainless steel structural frame member, and 22b of the outer cover 22. The middle jutting portion 59 (shown on FIG. 1) comprises middle jutting portion 38 on the inner cover member 34, middle jutting portion 31 on stainless steel structural frame member 27, and middle jutting portion 25 on insulative outer cover 22 (shown on FIG. 3). The end jutting portion 60 (shown on FIG. 1) comprises end jutting portion 39 on inner member cover 34, end jutting portion 32 on stainless steel structural frame member 27, and end jutting portion 26 on outer cover member 22 (shown on FIG. 3). Inner cover member 34 and insulative outer cover 22 both terminate at the jaw connection point 60a (not shown) at the end of jutting portion 60. The stainless steel structural frame member 27 through the end jutting portion 60 between the cover members and emerges from the enclosed insulative cover as the leading tapered edge 33.

The leading tapered edge 33 of stainless steel structural frame member 27 features a platypus tapered profile with marking indicator lines 52 displayed at its exposed end 33d and clamp stop 64 which, when the apparatus is fully closed, touches top jaw member 53 at clamp stop 63. The insulative bottom jaw member 49 slides over the tapered leading edge 33 and abuts the cover member (comprised of outer cover member 34 and inner cover member 22) at end jutting portion 60 (shown on FIGS. 1 and 5). The bottom jaw member 49 exposes the leading tapered edge 33d on the non-clamping side with marking lines 52 showing (not shown). On the clamping side of bottom jaw member 49 is an electrode 51. The electrode 51 inserts into the bottom jaw 49 in electrode holding cavity 50. The electrode 51 has an inlay 70 (see FIG. 7) where wire 47 attaches and touches it, providing to the electrode RF voltage. The marking indicator lines 52 correspond to the beginning, middle, and end of the electrode 51. The marking indicator lines 52 are located on both side walls of the bottom jaw member 49 and on the bottom of the leading tapered edge 33d of the stainless steel structural frame member 27.

Both conductor wires 47 and 48 enter the bipolar modular forceps assembly at cylindrical entrance passageway 58 of upper arm 21 located below the digit insertion ring 37 and extend through the passageway between mating set of cover members 34 and 22 and the stainless steel structural frame member 27 of the upper arm 21 until the conductor wires reach the pivot joint 43. Wire 47 is insulated as it runs along and between the inner surface 27c of the member 27 and the inner surface 34c of the cover member 34. Wire 47 is insulated throughout the apparatus except at 47b, where the wire attaches to connector cable 77a through the joint insulator, heat shrink tubing 76 and at end 47a, where the wire connects to the electrode. Similarly, wire 48 is insulated as it runs along and between the inner surface 27c of the member 27 and the inner surface 34c of the cover member 34. Wire 48 is insulated throughout the apparatus except at 48b, where the wire attaches to connector cable 77a through the joint insulator, heat shrink tubing 77 and at end 48a, where the wire connects to the electrode. When wires 47 and 48 reach bulging portions 34b and 27b, they run along and in the wire guide 78 of the stainless steel structural frame member 27 (see FIG. 5). Conductor wire 47 continues over the pivot joint to bottom jaw 49 and connects to the inlay of electrode 51. Conductor wire 48 wraps around the pivot pin 41 (illustrated on FIG. 5) goes through the inner cover members, first 34, then 14, and proceeds out from the end jutting portion 60 along the leading tapered edge to the top jaw 49 and connects to the inlay 56 of electrode 55.

The upper 21 and lower arm 1 are connected by running a pin through first the stainless steel frame member 7, then the inner cover member 14, then the inner cover member 34 and finally the stainless steel frame member 27. The inner cover members 16 and 34 together such that their exterior surfaces 34d and 14d face each other, the arm stops 68a and 69a fit into the opposing arm stop guides 69 and 68, and the pivot gaps 43a and 43b align (see FIG. 3). Then, the stainless steel structural frame member 7 is fitted into the U-shape inner surface 14c of the cover member 14 so that the bulging portions 7b and 14b fit snugly and the elongated portions 7a and 14a fit to fully cover side 7c of the stainless steel frame member Then, the stainless steel structural frame member 27 is fitted in the U-shape inner surface of the cover member 34 so that the bulging portions 27b and 34b fit snugly and the elongated portions 27a and 34a fit to fully cover side 27c of the stainless steel frame member. The wires 47 and 48 run between the inner cover member 34 and the stainless steel structural frame member 27 of the upper arm. FIG. 4 shows a cross section at the center point of the pivot joint 43, in the middle of the offset bulge 21b and 1b, looking towards the bottom jaw member 49 and top jaw member 53. The modular construction of each arm is visible with the upper arm 21 comprising outer cover member 22 and inner cover member 34 enclosing the stainless steel structural frame member 27 through lip 24 and lip 40. The lower arm 1 comprises of outer cover member 2 and inner cover member 14. Cover 2 comprises an interior surface 2c and an outer surface 2d. Cover 14 comprises an interior surface 14c and an outer surface 14d. The two covers enclose the stainless steel structural frame member 7 through lip 4 and lip 20 with their respective inner surfaces, 2c and 14c, facing inward and toward the stainless steel structural frame member. The two covers connect to form seam 82 (see FIG. 2) The pin 41 enters from stainless steel structural frame member 7, with the pin shoulder 41a at end 46 inserted first into stainless steel frame member 7 at hole 9 on side 7d. The pin passes through inner cover member 14, through inner cover member 34 and is pushed through stainless steel frame member 27 at hole 29 on side 27c so that the pin shoulder 41a pops through and rests in countersink 10a. The pin shoulder 41a and the pin head 44 should be spaced so that the two arms are free to rotate with only the arm stops as a limitation. Conductor wires 47 and 48 run along the inside of upper arm 21, along the inner cover member 34 and the inner side of stainless steel structural frame member 27. Wire 48 wraps around the pin bushing 42 beginning at the top of the bushing making one full revolution so that the wire exits the bushing emerging in the direction of the clamping side of the assembly. Pin 41 anchors the two stainless steel arms together, with outer cover member 2 covering the pin head 44 and outer cover member 22 covering the 46 end of pin 41. The outer cover members 14 and 2 are connected to each stainless steel structural frame member by connecting the male connectors 23 and 3 placed on the inner surface 22c and 2c of the outer cover members directly to the female receptors 23 and 8 of the stainless steel structural frame member and then snapped into the female receptors 36 and 16 of the inner cover members. A glue or sealant may be added to the seam 83 and 82 between the inner and outer cover members to add rigidity and prevent fluids from entering the cavities formed by the cover members.

FIG. 5 shows an angular side profile that illustrates how the wires emerge from pivot joint 43. Conductor wire 47 extends from the upper arm 21 and passes over the top of pivot joint 43 into the bottom jaw 49. Conductor wire 48 extends from the upper arm 21 and wraps around the pin 41, making one complete revolution before emerging into the top jaw 53. By wrapping conductor wire 48 around the pin, the conductor wires are contained for added safety by ensuring the wires remain inside the assembly and are not easily pulled out or dislodged from the electrodes.

The inner cover members of each arm comprise an arm stop with an arm stop guide. On the cover of lower arm 14 the arm stop 69a is comprised of a solid cylindrical member located on the inner side of the cover and placed beside the pivot joint 43 proximal to the middle jutting portion 18. Below the lower arm stop on the lower arm is the arm stop guide 69 which is comprised of an arch-shaped gap in the lower arm with a width to accept the arm stop of the upper arm and allow the stop to slide within it as the lower arm is rotated about the pivot joint 45. The upper arm stop 68a is comprised of a male cylindrical member located on the inner side of the cover and placed beside the pivot joint proximal to the stainless steel middle jutting portion 38. Above the arm stop, the arm stop guide 68 is located and comprised of a gap with a width to accept the arm stop of the upper arm and allow the stop to slide within it as the upper arm is rotated about the pivot joint 45. When the upper and lower arms are rotated about the pivot joint 43 and placed in the open configuration, the arm stops rotate towards each other traveling along their guides. When the arm stops meet, they prevent further separation of the arms and jaws. The arm stops provide a maximum separation for the top and bottom jaws and prevent damage and breakage to the forceps assembly.

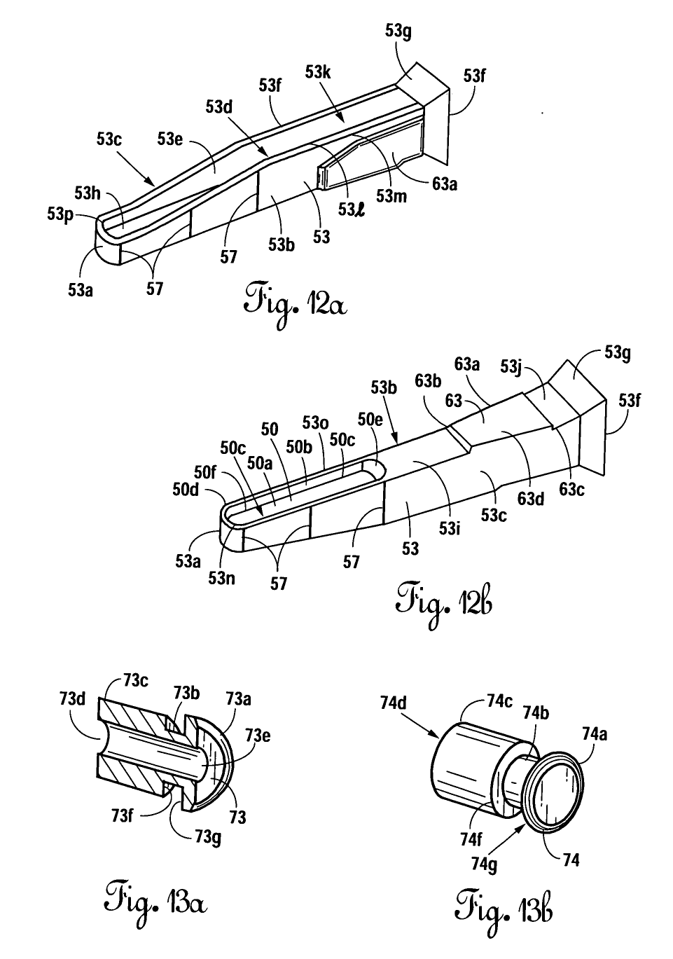

The assembly comprises two plugs, preferably made from rubber or other flexible material. The wire side cable plug 73 is comprised of a cylindrical member 73c, a grasping indentation 73b and a plug cap 73a (see FIG. 13a). A cylindrical cable passageway runs through the members 73a, 73b, and 73c to allow the cable to pass through the plug and into the rest of the assembly and attach to the exposed wires 48 and 47. The plug is inserted into the cylindrical passageway 58 such that the indentation grasps the plug grasp 22e (FIG. 10) and 34e (not shown). The plug 73c forms a seal with the cable and also seals the rest of the arm structure. The non-wire side plug 74 is similarly sized to 73. It is comprised of a cylindrical member 74c, a grasping indentation 74b and a plug cap 74a (see FIG. 13b). The plug is inserted into cylindrical passageway on the non-wire carrying arm such that the plug grasps 14e and 2e fit into the indentation on the plug 74b. This plug seals the end of the arm and prevents contaminants from entering the cavities formed by the connection of the cover members.

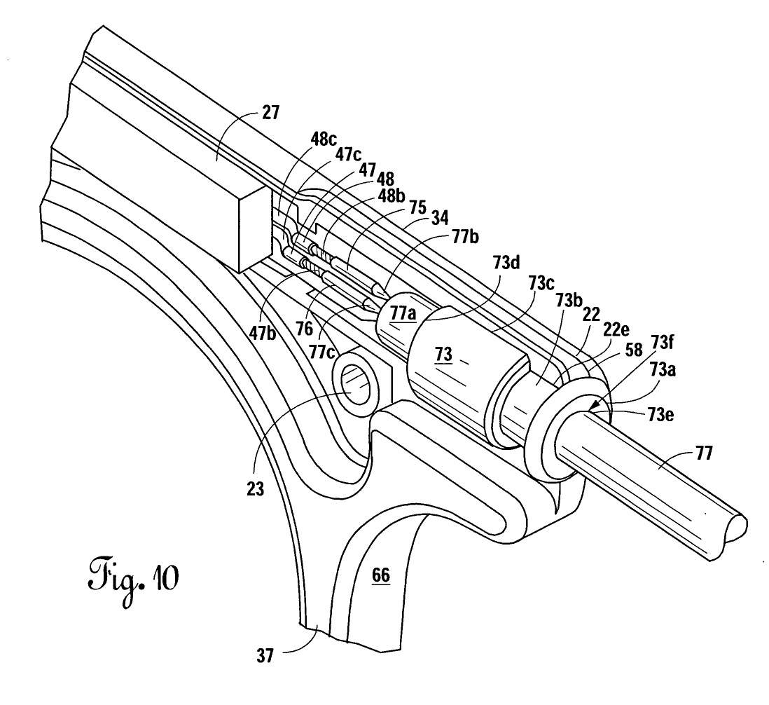

FIG. 10 illustrates the connection between the wires 47 and 48 and the cable 77 leading to the disposable connector 79. The wires 47 and 48 emerge from the cavity between the stainless steel structural frame member 27 and the inner cover member of the upper arm and enter the cylindrical entrance passageway. Each wire 47 and 48 having an exposed end 47b and 48b is connected by the joint insulators and heat shrink tubing 76 and 75, respectively, to the cable exposed end 77a and 77b. The cable passes through the cable plug 73 and out to the connector 79 (FIG. 11).

FIG. 11 illustrates the components that comprise the connector. The elements include an interface 91, top shell 92, bottom shell 93, a bend relief 94, and internal components 95, 96, and 97. Cable 77 enters the bend relief 94 and passes through component 94f and passes to 94e. Ferrule 97 is inserted over cable end 77d. Chip 19 is attached to cable end 77 placing the chip into the mouth-piece 97a of the ferrule. Peek contact block 95 is attached to the pins of the chip 96 so that the connectors 95a point away from the chip. The top shell and bottom shell are bonded together matching edges 93c and 93d with 92c and 92d, respectively, to enclose the internal components so that the top shell ends 93f and 92f rest against and are bonded to surface 94g of the bend relief. Interface 91 at end 91e is then inserted into the cavity formed by shell entrance 92e and 93e so that the connectors 95a fit into end 91e. Interface ring 91b is bonded to the shell ends 93f and 92f to seal all internal components between the bend relief 94 and the interface 91.

Before concluding, it is to be understood that the terminology employed in this application is for the purpose of describing particular embodiments. Unless the context clearly demonstrates otherwise, it is not intended to be limiting. In this specification and the appended claims, the singular forms “a,” “an” and “the” include plural references unless the context clearly dictates otherwise. Conversely, it is contemplated that the claims may be drafted to exclude any optional element or be further limited using exclusive terminology as “solely,” “only” and the like in connection with the recitation of claim elements or by use of a “negative” limitation. It is also contemplated that any optional feature of the inventive variations described herein may be set forth and claimed independently, or in combination with any one or more of the features described herein.

Although the foregoing specific details describe various embodiments of the invention, persons reasonably skilled in the art will recognize that various changes may be made in the details of the apparatus of this invention without departing from the spirit and scope of the invention as defined in the appended claims. Therefore, it should be understood that, unless otherwise specified, this invention is not to be limited to the specific details shown and described herein.

We claim:

1) A bipolar modular forceps assembly for tissue treatment comprising:
upper and lower opposing structural frame member scissor arms that are pivotally connected through a pivot joint;
mating opposed cover members enclosing each of the opposing scissor arms and including digit insertion rings at one end;
opposing jaw members with opposing electrodes mounted on the arms on the other side of the pivot point away from the rings;
conductor wires extending through a passageway formed by the one of the mating sets of opposed cover members, passing through a pivot joint, and passing through a passageway formed in the jaw members with a respective one of the conductor wires connected to each of the electrodes.

2) The bipolar modular forceps assembly as claimed in claim 1 wherein one of the wires extending through the passageway formed by one of the mating sets of opposed cover members extends around the pivot joint to hold the conductor wires in place.

3) The bipolar modular forceps assembly as claimed in claim 1 wherein the cover members are formed of molded material.

4) The bipolar modular forceps assembly as claimed in claim 1 wherein the scissor arms are formed of structural metal and enclosed by the cover members.

5) The bipolar modular forceps assembly as claimed in claim 1 further comprising a locking mechanism to maintain clamping force on tissue.

6) The bipolar modular forceps assembly as claimed in claim 1 further comprising insulation on the conductor wires.

7) The bipolar modular forceps assembly as claimed in claim 1 further comprising a pivot pin pivotally securing the upper and lower opposing structural frame member scissor arms at the pivot joint.

8) The bipolar modular forceps assembly as claimed in claim 1 wherein the cover members are formed of molded insulative material.

9) The bipolar modular forceps assembly as claimed in claim 1 wherein the cover members are formed of insulative material to provide insulation to the electrodes and to a user and surrounding tissue, allowing voltage to only travel between electrodes and tissue being grasped by the electrodes.

10) A bipolar modular forceps assembly for tissue treatment comprising:
upper and lower opposing structural frame member scissor arms formed of structural metal and pivotally connected through a pivot pin pivotally securing the upper and lower opposing structural frame member scissor arms at the pivot join;
mating opposed cover members enclosing each of the opposing scissor arms and including digit insertion rings at one end and said cover members being formed of molded insulative material to provide insulation to the electrodes and to a user and surrounding tissue, allowing voltage to only travel between electrodes and tissue being grasped by the electrodes and having a locking mechanism to maintain clamping force on tissue;
opposing jaw members with opposing electrodes mounted on the arms on the other side of the pivot point away from the rings
insulated conductor wires extending through a passageway formed by the mating sets of opposed cover members and passing through a passageway formed in the jaw members with a respective one of the conductor wires connected to each of the electrodes;
one of the wires extending through the passageway formed by one of the mating sets of opposed cover members extends around the pivot joint to hold the conductor wires in place.
